(19) United States
(12) Patent Application Publication
(10) Pub. No.: US 2006/0205730 A1
(43) Pub. Date: Sep. 14, 2006

(54) Discovery of valuable properties for the primary energy source "PES 2000 plus folic acid"

(76) Inventor: William Hubert Livingston, Artesia, NM (US)

Correspondence Address: Livingston Research Corporation, R223 North 13th Street, Artesia, NM, 88210, US

(21) Appl. No.: 11/138,086

(22) Filed: Mar. 11, 2005

(51) Int. Cl. A61K 31/198; A61K 31/525; A61K 31/19

(52) U.S. Cl. 514/251; 514/574; 514/566

(57) ABSTRACT

1. Increased energy is produced by stimulating the Citric Acid Cycle, in 1957 Dr. Livingston successfully treated Pregnancy Toxemia in sheep, with intravenous oxaloacetic acid, demonstrating that Krebs Cycles can be stimulated by intravenous injection. 2. U.S. Pat. No. 5,475,031, “DISCOVERY OF A VALUABLE PROPERTY FOR SUCCINIC ACID AND OTHER IMTERMEDIARY METABOLITES”, demonstrated that oral intake of Citric Acid Metabolites stimulates the Citric Acid Cycle. 3. The following actions; that are valuable happened when an oral therapeutic dose of “PES 2000 plus Folic Acid” are, administered to animals or humans. Succinic Dehydrogenises (Co Enzyme Q 10) supplementation may be needed if it is deficient in an animal, or human. A. An oral therapeutic dose of “PES 2000 plus Folic Acid”, reduces formation of triglycerides, cholesterol and apolipoprotein. This reduction helps prevent Alzheimer's disease, and heart attacks in humans and animals. B. An oral therapeutic dose “PES 2000 plus Folic Acid” has been shown to be effective in preventing damage caused by diabetes, in humans and animals”. 1). Decreases (damaging) negative free radicals (O negative), that damages the blood vessels of the eyes, brain and etc. (microangiopathy). Decreases negative free radicals that can cause blood clots in extremities and the heart (macroangiopathy}. 2). Lowers blood sugar, triglycerides, cholesterol, A1c, apolipoprotein levels, in type one and two diabetes, and reduces insulin resistance in animals and humans. C. An oral therapeutic dose of calcium succinate when administered orally to humans and animals, increases bone and tissue calcium absorption and prevents milk fever. D. It has been discovered that a therapeutic dose of intermediary metabolites when taken by athletes, are able to stay in condition with less exercise. This is especially valuable in athletes who have been injured and must rest or lay out.

### BACKGROUND OF THE INVENTION

Method of discovery is as follows: “Primary Energy Source, “PES 200 Plus Folic Acid”, containing a therapeutic dose, when taken by individual humans and animals, orally, did the following:

A. Increased energy by stimulating the energy cycle, the “Citric Acid Cycle”, and slowed aging in humans and animals. This has been demonstrated in thousands of humans, horses, cats and dogs. This was demonstrated thru the use of Livingston Animal Clinic's. SR-38, a precursor of “PES 2000”

B. Research work shows “PES 2000 plus Folic Acid” will help prevent Alzheimer's disease.

C. A therapeutic dose of “PES 2000 plus Folic Acid” has been shown effective in preventing damage caused by diabetes, thru research conducted by Livingston Research Corporation (LRC) and Livingston Animal Clinic

D. A therapeutic dose of “PES 2000 plus Folic Acid” has a protein sparring ability.

1. A therapeutic dose of “PES 2000 PLUS FOLIC ACID” aids in the Prevention of damage caused by diabetes in humans and animals by lowering cholesterol, triglycerides, sugar levels and helps prevent insulin resistance.

2. A therapeutic dose of Citric Acid Metabolites and Calcium, will help Prevent ketosis and milk fever in humans and animals. The type of calcium may be varied. It may be administered either intravenously or orally.

3. A therapeutic dose of “PES 2000 PLUS FOLIC ACID” or succinic acid and some of the other Kreb Cycle Metabolites will over come Hepatic Coma from over indulgence of alcohol when taken orally.

4. A therapeutic dose of “PES 2000 PLUS FOLIC ACID”, helps prevent Alzheimer's disease in humans.
